(19) United States
(12) Patent Application Publication
(10) Pub. No.: US 2022/0088767 A1
(43) Pub. Date: Mar. 24, 2022

(54) Personal Augmentation Suit and Method for Assisted Human Motion

(71) Applicant: SpringActive, Inc., Tempe, AZ (US)

(72) Inventors: Matthew A. Holgate, Mesa, AZ (US); Jeffrey A. Ward, Phoenix, AZ (US); Pahul Singh, Mesa, AZ (US); Garrett Repp, Gilbert, AZ (US)

(73) Assignee: SpringActive, Inc., Tempe, AZ (US)

(21) Appl. No.: 17/457,598

(22) Filed: Dec. 3, 2021

Related U.S. Application Data
(63) Continuation of application No. 16/143,322, filed on Sep. 26, 2018.
Application No. 16/143,322 is a continuation-in-part of application No. 15/674,386, filed on Aug. 10, 2017, now Pat. No. 10,772,785.
(60) Provisional application No. 62/403,596, filed on Oct. 3, 2016.
(60) Provisional application No. 62/477,797, filed on Mar. 28, 2017.

(51) Int. Cl. B25J 9/00; B25J 9/10; A61H 1/00; A61H 3/00; A61F 5/02; A62B 35/00

(52) U.S. Cl. CPC B25J 9/0006; B25J 9/104; A62B 35/0018; A61H 3/00; A61F 5/02; A61H 1/00

(57) ABSTRACT

A human motion assistance device has an upper torso harness for attaching to a user. The upper torso harness has a backpack assembly and shoulder strap, which buckles in front of the user. The upper torso harness can be IOTV. Leg straps are affixed to the upper torso harness proximate to a gluteal area of the user and configured to extend down a hamstring area to a knee pad and further extend down a tibia area. The knee pad opens along a vertical or angled segment and the calf portion opens over the calf. Seat webbing is connected to leg straps for additional support. Upper torso harness and leg straps are made with a combination of durable materials such as textured nylon, polyurethane coated polyester, as well as elastic material and webbing material. The upper torso harness and leg straps are passive without active members.

### CLAIM TO DOMESTIC PRIORITY

The present application is a continuation of U.S. application Ser. No. 16/143,322, filed Sep. 26, 2018, which is a continuation-in-part of U.S. application Ser. No. 15/674,386, now U.S. Pat. No. 10,772,785, filed Aug. 10, 2017, which claims the benefit of U.S. Provisional Application No. 62/403,596, filed Oct. 3, 2016, and U.S. Provisional Application No. 62/477,797, filed Mar. 28, 2017, which applications are incorporated herein by reference.

### FIELD OF THE INVENTION

The present invention relates in general to a human assistance device, and more particularly, to a personal augmentation suit and method for assisted human motion.

### BACKGROUND OF THE INVENTION

A variety of human assistance devices provide aid for human motion. Some work tasks and activities demand frequent and heavy lifting or long-term strenuous movement, beyond normal unassisted physical exertion, and can lead to exhaustion and potential injury. In particular, excessive strain on muscles and joints can cause injury to the back, legs, and knees with associated reduction in productivity. The human assistance device as worn by the user is designed to overcome or compensate for the added load or strain and make many work tasks easier to perform.

In one embodiment, the human assistance device uses an exoskeleton with rigid components e.g., linkages and joints, attached to the user's body. The exoskeleton joints are ideally positioned to have an axis of rotation collinear with a natural axis of rotation for adjacent joints. The rigid exoskeleton relies on a framework of linkages connected to the body at select locations via pads and straps to provide the ability to augment human movements that need assistance or otherwise enhance the user's performance, stability, balance, and safety. As the user flexes or extends his limbs, these rigid links move in parallel with the limb, adding considerable inertia to movement. Unfortunately, the rigid exoskeleton also causes considerable restriction to the user's motion that impedes natural and fluid movement.

In another example, U.S. patent publication 2015/0321339 discloses a soft exosuit that generates forces about one or more joints based on anchor elements and connection elements disposed between the anchor elements. The exosuit uses sensors to determine forces on the anchor or connection elements. Actuators are configured to change tension in the soft exosuit in response to the sensors. The exosuit tends to be complex with an overreliance on active components, such as sensors and actuators, to control its operation. The intricate interconnection of anchor elements, connection elements, sensors, and actuators tends to be expensive to manufacture, difficult to configure, slow in response, and overall low reliability.

### BRIEF DESCRIPTION OF THE DRAWINGS

FIGs. 1a-1c illustrate a user wearing a passive P2K suit;

FIGS. 2a-2b illustrate the user transitioning from a seated position to standing position with aid from the passive P2K suit;

FIGS. 3a-3b illustrate elastic bands in the passive P2K suit;

FIGS. 4a-4b illustrate a second embodiment of the passive P2K suit with elastic leg straps;

FIGS. 5a-5b illustrate a third embodiment of the passive P2K suit with front torso cross straps;

FIGS. 6a-6b illustrate a fourth embodiment of the passive P2K suit with buckles to adjust the upper torso harness;

FIG. 7 illustrates further detail of the leg straps and foot anchor;

FIGS. 8a-8c illustrate further detail of the leg straps and foot anchor;

FIGS. 9a-9b illustrate a fifth embodiment of the passive P2K suit with back support;

FIGS. 10a-10c illustrate a sixth embodiment of the passive P2K suit with a backpack assembly;

FIGS. 11a-11c illustrate a seventh embodiment of the passive P2K suit with a backpack assembly and split knee pad;

FIGS. 12a-12c illustrate an eighth embodiment of the passive P2K suit with a backpack assembly, split knee pad, and calf cuffs;

FIG. 13 illustrates the split knee pad and calf cuffs in the open position; and

FIGS. 14a-14b illustrate a ninth embodiment of the passive P2K suit with an IOTV upper torso assembly.

### DETAILED DESCRIPTION OF THE DRAWINGS

The present invention is described in one or more embodiments in the following description with reference to the figures, in which like numerals represent the same or similar elements. While the invention is described in terms of the best mode for achieving the invention's objectives, those skilled in the art will appreciate that the description is intended to cover alternatives, modifications, and equivalents as may be included within the spirit and scope of the invention as defined by the appended claims and their equivalents as supported by the following disclosure and drawings.

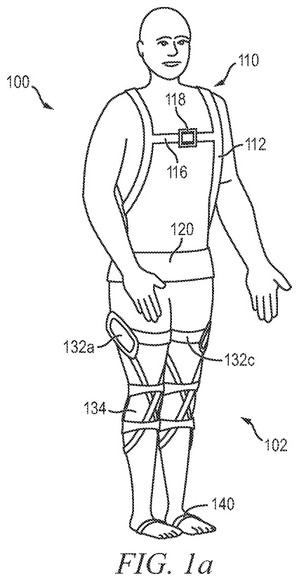

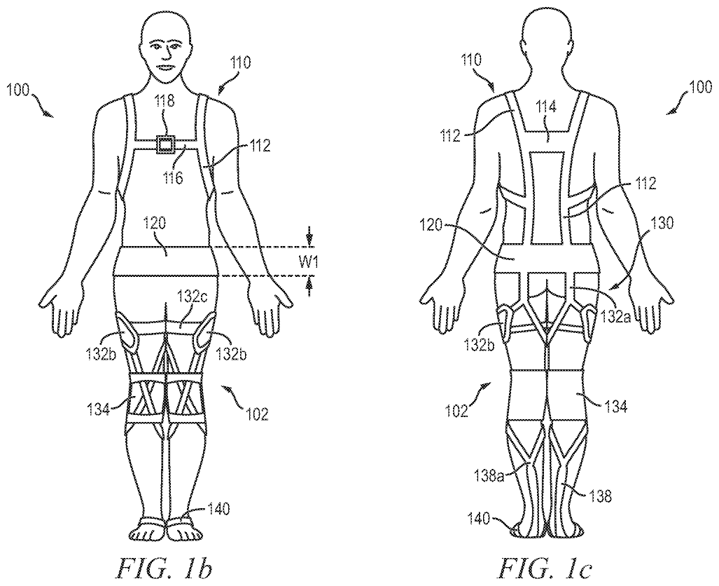

FIG. 1a shows user 100 wearing potential to kinetic suit (P2K) 102 suitable for human assistance in crouching, squatting, and lifting under load. P2K 102 is a passive personal augmentation suit or strapping system, i.e., no active components, applicable to many types of human activity and physical work, such as moving heavy articles, up and down motion under load, and other repetitive, long-term physical exertion. P2K 102 is capable of storing potential energy in the strapping arrangement, e.g., potential energy is stored in elastic strapping when transitioning from a standing position to a crouched or squatting position. The potential energy in P2K 102 is released when user 100 moves from the crouched or squatting position to return upright in stance, thereby assisting the user in the motion, particularly while under load. P2K 102 increases endurance, reduces fatigue, and decreases potential for injury associated with such activity, particularly for the user's back, legs, and knees. P2K 102 includes an interconnected arrangement of straps, belts, and braces to provide passively loaded support and reaction for user 100 while assisting with human motion under load. FIG. 1b shows a front view of P2K 102; FIG. 1c shows a back view of P2K 102.

Upper torso harness 110 includes shoulder straps 112 around the shoulders of user 100, back interconnect strap 114 between shoulder straps 112, front strap 116 with adjustable buckle 118. Back interconnect strap 114 and front strap 116 are affixed to shoulder straps 112 with sewing, rivets, belt, buckles, buttons, snaps, hooks, adhesive, Velcro, or other secure attachment mechanism. Shoulder straps 112 extend down the back of user 100 to waist and back belt 120. Waist and back belt 120 wraps around the waist, hips, and lower back area and secures with Velcro or buckles. In one embodiment, belt 120 has a width W1 of 7-18 centimeters (cm). Shoulder straps 112 can slide within upper torso harness 160 and are affixed to belt 120 with sewing, rivets, belt, buckles, buttons, snaps, hooks, adhesive, Velcro, or other secure attachment mechanism. Waist and back belt 120 provides a brace to support the lower back and tightens to squeeze the hips when squatting.

Thigh straps 130 extend from waist and back belt 120 over the gluteal area and around the thigh area of user 100. Thigh straps 130 connect to belt 120 with sewing, rivets, belt, buckles, buttons, snaps, hooks, adhesive, Velcro, or other secure attachment mechanism. In particular, thigh straps 130 include Y-configured strap 132a over the gluteal area and hamstrings, side straps 132b, and front straps 132c over the quadriceps. Knee support 134 connects to Y-configured strap 132a and side straps 132b with sewing, rivets, belt, buckles, buttons, snaps, hooks, adhesive, Velcro, or other secure attachment mechanism. Knee support 134 includes a pocket or cavity for the knee of user 100, with support material covering the knee and straps around the knee. Knee support 134 and side straps 132b can include pads to provide hard support points for P2K 102. Calf straps 138 connect to knee support 134 with sewing, rivets, belt, buckles, buttons, snaps, hooks, adhesive, Velcro, or other secure attachment mechanism. In particular, calf straps 138 include Y-configured strap 138a over the calf of user 100. Foot anchor 140 connects to calf strap 138 with sewing, rivets, belt, buckles, buttons, snaps, hooks, adhesive, Velcro, or other secure attachment mechanism. Foot anchor 140 is configured to connect to a shoe, boot, or directly to the foot, e.g., under and over the foot with an adhesive or sock-like slip-on.

P2K 102 is modular in construction, configuration, and use. P2K 102 can be configured with various combinations of components described in FIGS. 1a-1c. For example, P2K 102 can be used as waist and back belt 120 with thigh straps 130, knee support 138, calf straps 138, and foot anchor 140, without shoulder straps 112. Configurations with shoulder straps 112 have the advantage of encouraging good posture. The various straps and belts in FIGS. 1a-1c can be implemented using fabric or leather material, elastic bands, cables, or other suitable flexible connecting members. Springs, pulleys, guides, and other compliant members can be used in place of or to augment the straps and belts within P2K 102. P2K 102 is adjustable for size and pre-tensioning the elastic components.

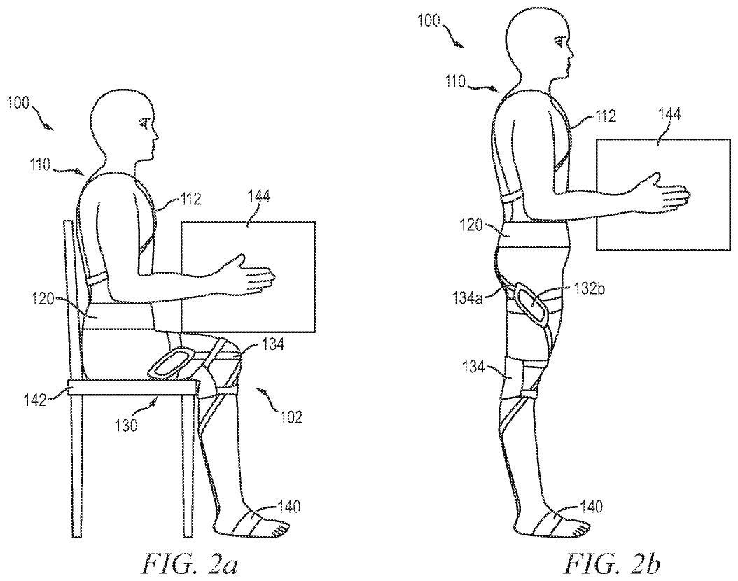

Consider user 100 wearing P2K 102 in the seated position on seat 142 and holding load 144 in FIG. 2a. P2K 102 stores potential energy in the strapping arrangement, e.g., the elastic strapping stretches and stores potential energy when transitioning from a standing position to a crouched, squatting, or seated position. The potential energy in P2K 102 remains available while user 100 is seated to help with the opposing motion, i.e., standing up under load. Waist and back belt 120 provides a brace to support the lower back and tightens to squeeze the hips when sitting. User 100 stands up from the seated position with load 144, as shown in FIG. 2b. The potential energy in P2K 102 is released when user 100 moves from the crouched, squatting, or seated position to return upright in stance, thereby assisting the user in the motion, particularly while under load. P2K 102 reduces the effort, strain, fatigue, and potential for injury associated with such movement in physical labor work environments. A similar concept applies to user 100 standing from the kneeling or crouching position. In one embodiment, knee support 134 includes a torsional spring to aid with transition from a seated, kneeling, or squatting position to a standing position, without the use of active components. Alternatively, knee support 134 uses elastic bands, or a combination of non-elastic and elastic bands, to assist user 100 in the sit to stand transition.

In general, P2K 102 utilizes a flexible, modular, passive strapping system that, in one embodiment, extends from upper torso harness 110 to waist and back belt 120 and further travels down gluteal area and hamstrings with thigh straps 130 to knee support 134. P2K 102 can further extend from knee support 134 over the calf area with calf straps 138 to foot anchor 140. P2K 102 gains support from upper torso harness 110 and belt 120, as well as the strapping arrangement from the back of the thigh, front of the knee, and back of the tibia to anchor at the foot. Because the straps cross at the waist and over the length of the leg, the strapping arrangement stores energy in the elastic bands or springs during the squatting or crouching motion to be released during transition to the standing position thus assisting with human motion under load. The crossed straps also hold P2K 102 in place during various movements, such as squatting.

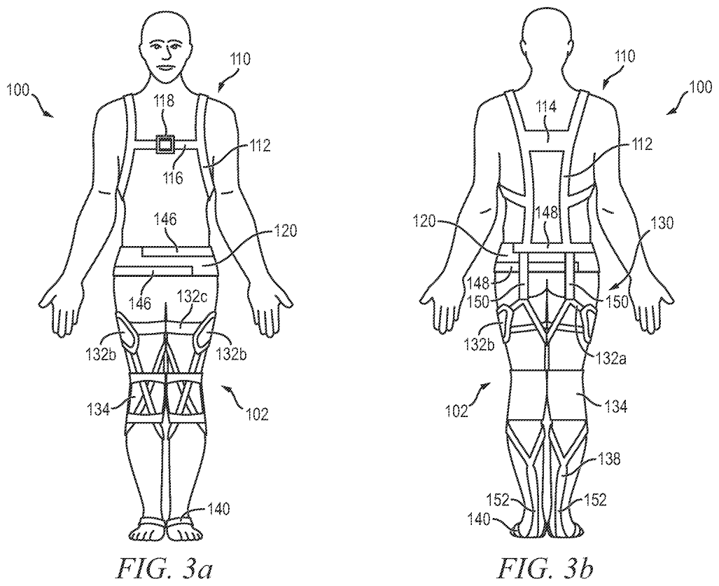

Elastic bands have been found to be particularly effective in areas 146 and 148 (belt 120), area 150 (thigh straps 130), and area 152 (calf straps 138) for assisting with human motion under load, as shown in FIGS. 3a-3b. Elastic bands in areas 146-152 can vary in elasticity and stiffness with any preloading adjustable for the user 100 during the fitting process.

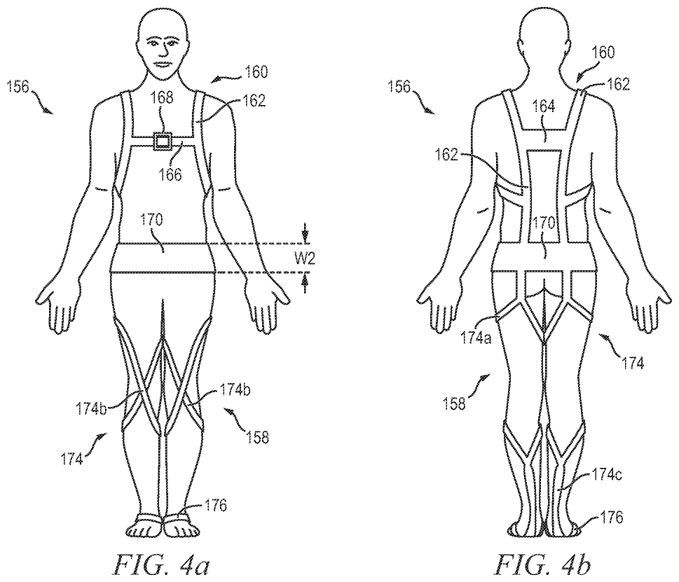

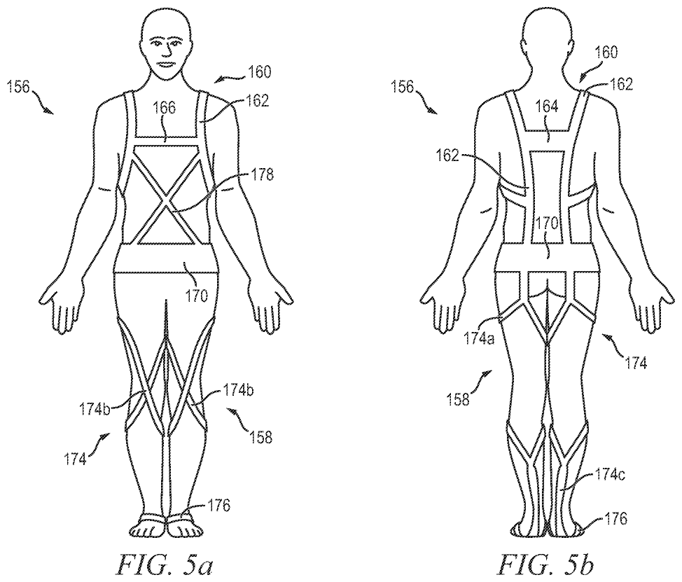

FIGS. 4a-4b illustrate another embodiment of user 156 wearing P2K 158 suitable for human assistance in crouching, squatting, and lifting under load. FIG. 4a shows a front view of P2K 158; FIG. 4b shows a back view of P2K 158. P2K 158 is a passive personal augmentation suit or strapping system, i.e., no active components, applicable to many types of human activity and physical work, such as moving heavy articles, up and down motion under load, and other repetitive, long-term physical exertion. P2K 158 is capable of storing potential energy in the strapping arrangement, e.g., potential energy is stored in elastic strapping when transitioning from a standing position to a crouched or squatting position. The potential energy in P2K 158 is released when user 156 moves from the crouched or squatting position to return upright in stance, thereby assisting the user in the motion, particularly while under load. P2K 158 increases endurance, reduces fatigue, and decreases potential for injury associated with such activity, particularly for the user's back, legs, and knees. P2K 158 includes an interconnected arrangement of straps, belts, and braces to provide passively loaded support and reaction for user 156 while assisting with human motion under load.

Upper torso harness 160 includes shoulder straps 162 around the shoulders of user 156, back interconnect strap 164 between shoulder straps 162, front strap 166 with adjustable buckle 168. Back interconnect strap 164 and front strap 166 are affixed to shoulder straps 162 with sewing, rivets, belt, buckles, buttons, snaps, hooks, adhesive, Velcro, or other secure attachment mechanism. Shoulder straps 162 extend down the back to waist and back belt 170. Waist and back belt 170 wraps around the waist, hips, and lower back area and secures with Velcro or buckles. In one embodiment, belt 170 has a width W2 of 7-18 cm. Shoulder straps 162 can slide within upper torso harness 160 and are affixed to belt 170 with sewing, rivets, belt, buckles, buttons, snaps, hooks, adhesive, Velcro, or other secure attachment mechanism. Waist and back belt 170 provides a brace to support the lower back and tightens to squeeze the hips when squatting.

Leg strap arrangement 174 extends down the legs of user 156. Leg straps 174 include Y-configured thigh portion 174a over the gluteal area and hamstrings of user 156, cross-configured knee portion 174b over the front of the knee area, and Y-configured calf portion 174c over the calf area. Thigh portion 174a connect to waist and back belt 170 with sewing, rivets, belt, buckles, buttons, snaps, hooks, adhesive, Velcro, or other secure attachment mechanism. Leg straps 174 utilize the bending motion of the ankle and knee for maximum stretching of the elastic bands. Foot anchor 176 connects to calf portion 174c behind the heel with sewing, rivets, belt, buckles, buttons, snaps, hooks, adhesive, Velcro, or other secure attachment mechanism. Foot anchor 176 connects to a shoe, boot, or directly to the foot, e.g., under and over the foot with an adhesive or sock-like slip-on.

P2K 158 is modular in construction, configuration, and use. For example, P2K 158 can be used as waist and back belt 170 with leg straps 174, without shoulder straps 162. The various straps and belts in FIGS. 4a-4b can be implemented using fabric or leather material, elastic bands, cables, or other suitable flexible connecting members. P2K 158 stores potential energy in the strapping arrangement, e.g., potential energy is stored in elastic strapping when transitioning from a standing position to a crouched, squatting, or seated position. The potential energy in P2K 158 is released when user 156 moves from the crouched, squatting, or seated position to return upright in stance, thereby assisting the user in the motion, particularly while under load. P2K 158 is adjustable for size and pre-tensioning the elastic components.

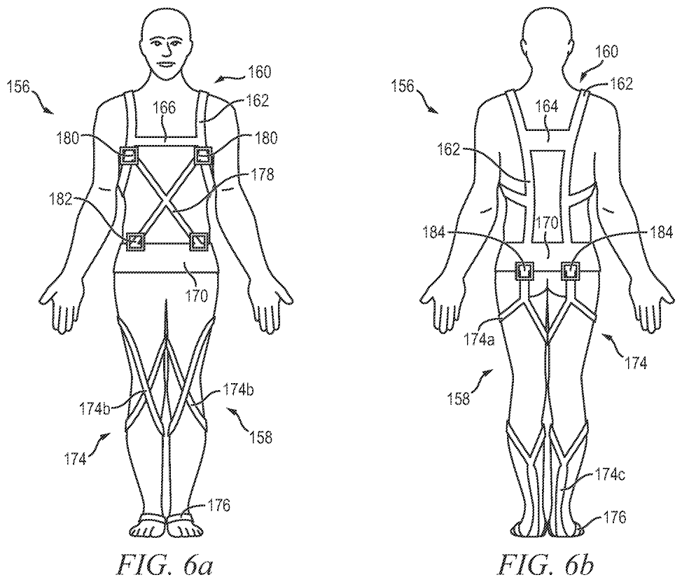

FIGS. 5a-5b illustrate a modified P2K 158, similar to FIGS. 4a-4b, with front torso cross straps 178 for additional support.

FIGS. 6a-6b illustrate a modified P2K 158, similar to FIGS. 4a-4b, with buckles 180 and 182 for adjustment and attachment points on the front of P2K 158, and buckles 184 for adjustment and attachment points on the back of P2K 158.

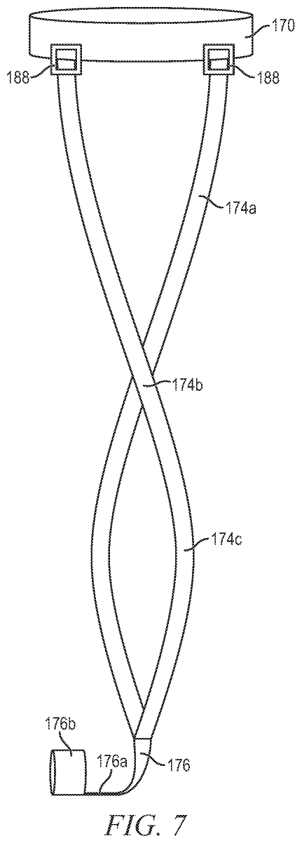

FIG. 7 shows further detail of leg straps 174 from waist and back belt 170 to foot anchor 176. Leg straps 174 can be made with elastic bands crossing at knee portion 174b, i.e., in front of the knee. Leg straps 174 can include buckles 188 or Velcro for ease of donning and doffing P2K 158. The user's foot slides over heel strap 176a and into arch wrap 176b to fix leg straps 174 in place. Foot anchor 176 can be attached over a shoe.

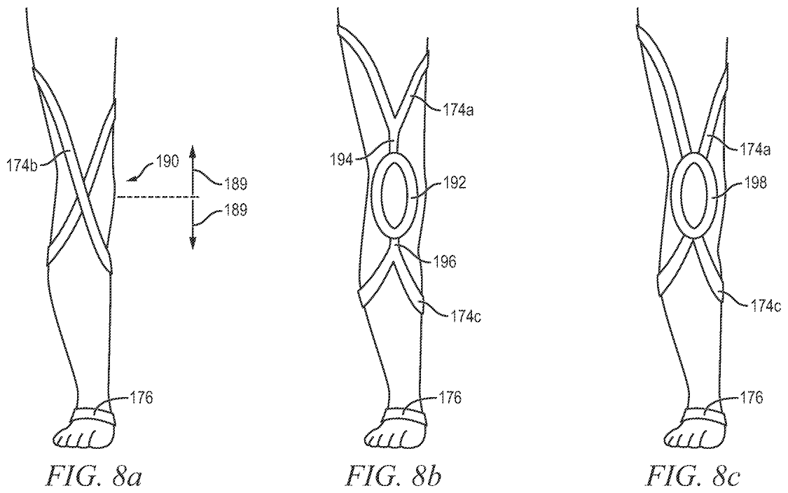

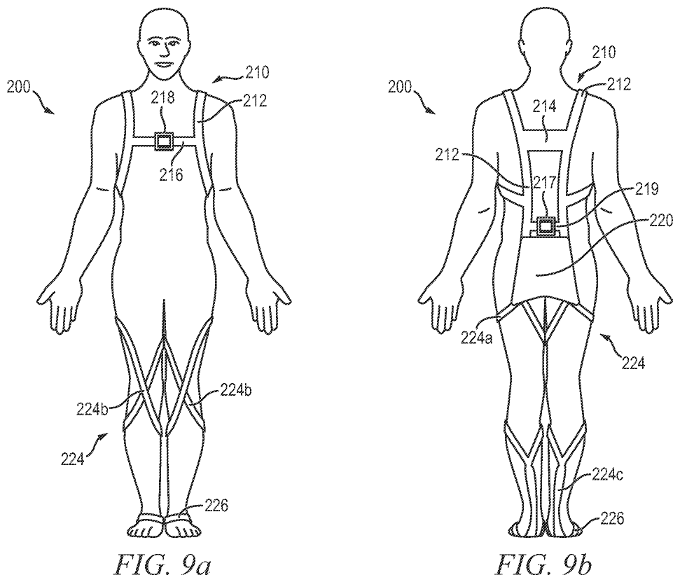

FIG. 8a shows leg straps 174 crossing in front of knee 190. Leg straps can be positioned to cross above, directly on, or below the knee cap, as shown by the range of arrows 189. FIG. 8b shows leg straps 174 crossing above and below the knee with pocket 192 around the knee cap for comfort and stability. A first single strap 194 extends above pocket 192 to thigh portion 174a. A second single strap 196 extends below pocket 192 to calf portion 174c. FIG. 8c shows leg straps 174 crossing in front of the knee with pocket 198 around the knee cap for comfort and stability. Thigh portion 174a connects to an upper point of pocket 198, and calf portion 174c connects to a lower point of pocket 198.

FIGS. 9a-9b illustrate another embodiment of user 200 wearing P2K 202 suitable for human assistance in crouching, squatting, and lifting under load. FIG. 9a shows a front view of P2K 202; FIG. 9b shows a back view of P2K 202. P2K 202 is a passive personal augmentation suit or strapping system, i.e., no active components, applicable to many types of human activity and physical work, such as moving heavy articles, up and down motion under load, and other repetitive, long-term physical exertion. P2K 202 is capable of storing potential energy in the strapping arrangement, e.g., potential energy is stored in elastic strapping when transitioning from a standing position to a crouched or squatting position. The potential energy in P2K 202 is released when user 200 moves from the crouched or squatting position to return upright in stance, thereby assisting the user in the motion, particularly while under load. P2K 202 increases endurance, reduces fatigue, and decreases potential for injury associated with such activity, particularly for the user's back, legs, and knees. P2K 202 includes an interconnected arrangement of straps, belts, and braces to provide passively loaded support and reaction for user 200 while assisting with human motion under load.

Upper torso harness 210 includes shoulder straps 212 around the shoulders of user 200, upper back interconnect strap 214 between shoulder straps 212, front strap 216 with adjustable buckle 218. Back interconnect strap 214 and front strap 216 are affixed to shoulder straps 212 with sewing, rivets, belt, buckles, buttons, snaps, hooks, adhesive, Velcro, or other secure attachment mechanism. Shoulder straps 212 extend down the back to back support 220 and affix with sewing, rivets, belt, buckles, buttons, snaps, hooks, adhesive, Velcro, or other secure attachment mechanism. Lower back interconnect strap 219 is connected above back support 220.

Leg strap arrangement 224 extends down the legs of user 200. Leg straps 224 include Y-configured thigh portion 224a over the gluteal area and hamstrings of user 200, cross-configured knee portion 224b over the front of the knee area, and Y-configured calf portion 224c over the calf area. Thigh portion 224a connects to back support 220 with sewing, rivets, belt, buckles, buttons, snaps, hooks, adhesive, Velcro, or other secure attachment mechanism. Leg straps 224 utilize the bending motion of the ankle and knee for maximum stretching of the elastic bands. Foot anchor 226 connects to calf portion 224c behind the heel with sewing, rivets, belt, buckles, buttons, snaps, hooks, adhesive, Velcro, or other secure attachment mechanism. Foot anchor 226 connect to a shoe, boot, or directly to the foot, e.g., under and over the foot with an adhesive or sock-like slip-on.

P2K 202 stores potential energy in the strapping arrangement, e.g., potential energy is stored in elastic strapping when transitioning from a standing position to a crouched, squatting, or seated position. The potential energy in P2K 202 is released when user 200 moves from the crouched, squatting, or seated position to return upright in stance, thereby assisting the user in the motion, particularly while under load.

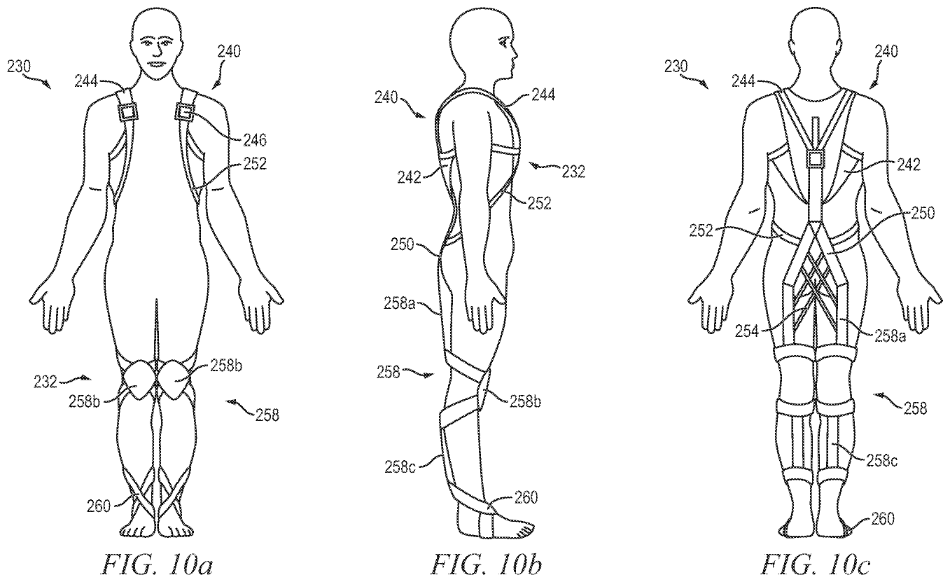

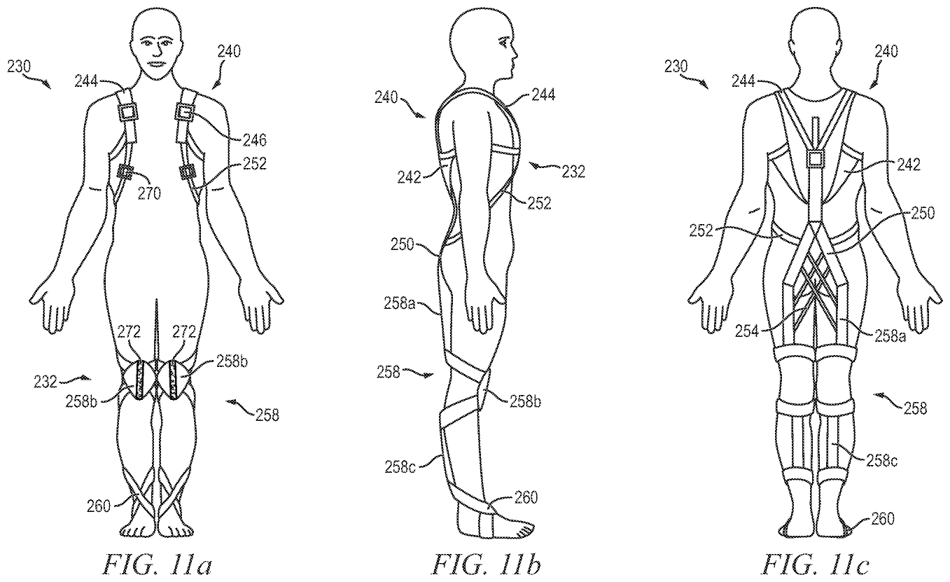

FIGS. 10a-10c illustrate another embodiment of user 230 wearing P2K 232 suitable for human assistance in crouching, squatting, and lifting under load. FIG. 10a shows a front view of P2K 232; FIG. 10b shows a side view of P2K 232; FIG. 10c shows a back view of P2K 232. P2K 232 is a passive personal augmentation suit or strapping system, i.e., no active components, applicable to many types of human activity and physical work, such as moving heavy articles, up and down motion under load, and other repetitive, long-term physical exertion. P2K 232 is capable of storing potential energy in the strapping arrangement, e.g., potential energy is stored in elastic strapping when transitioning from a standing position to a crouched or squatting position. The potential energy in P2K 232 is released when user 230 moves from the crouched or squatting position to return upright in stance, thereby assisting the user in the motion, particularly while under load. P2K 232 increases endurance, reduces fatigue, and decreases potential for injury associated with such activity, particularly for the user's back, legs, and knees. P2K 232 includes an interconnected arrangement of straps, belts, and braces to provide passively loaded support and reaction for user 230 while assisting with human motion under load. Portions of P2K 232 can be reflective material for safety.

Upper torso harness 240 includes backpack assembly 242 with shoulder straps 244. Backpack assembly 242 can be made with an air mesh material with openings to allow for air-flow on the back and reduces the overall weight. Backpack assembly 242 can include a combination of elastic material and webbing material. Backpack assembly 242 covers a portion of back area of user 230 to improve ergonomics and comfort, relieve lower back pressure, and ease of donning and doffing. Shoulder straps 244 extend over the shoulder and affix to backpack assembly 242 with sewing, rivets, belt, buckles, buttons, snaps, hooks, adhesive, Velcro, or other secure attachment mechanism. Shoulder straps 244 can slide relative to backpack assembly 242 for user comfort. Buckles 246 in shoulder straps 244 provide adjustment and attachment points. Buckles 246 can be used to adjust elastic pre-load (strength of the suit when squatting down) and enable P2K 232 to fit different body sizes. Back support 250 connects to backpack assembly 242 with sewing, rivets, belt, buckles, buttons, snaps, hooks, adhesive, Velcro, or other secure attachment mechanism. Side straps 252 extend from shoulder straps 244 to back support 250. Side straps 252 can include elastic material with buckles for adjustments and comfort, see FIG. 11a. Back support 250 slides or moves relative to backpack assembly 242 to minimize medial/lateral relative motion and provide pre-tensioning.

Leg strap arrangement 258 extends down the legs of user 230. Leg straps 258 include thigh portion 258a over the gluteal area and hamstrings of user 230, knee portion 258b over the front of the knee area, and calf portion 258c over the calf area. Thigh portion 258a connects to back support 250 with sewing, rivets, belt, buckles, buttons, snaps, hooks, adhesive, Velcro, or other secure attachment mechanism. Seat webbing 254 is connected to back support 250 and thigh portion 258a with sewing, rivets, belt, buckles, buttons, snaps, hooks, adhesive, Velcro, or other secure attachment mechanism. Seat webbing 254 is an elastic material for storing and releasing power and comfortable load distribution. Knee portion 258b includes a knee pad for knee protection while kneeling on the ground. Knee portion 258b anchors the knee when energy is being stored and returned by P2K 232. Leg straps 258 utilize the bending motion of the ankle and knee for maximum stretching of the elastic bands. Foot anchor 260 connects to calf portion 258c behind the heel with sewing, rivets, belt, buckles, buttons, snaps, hooks, adhesive, Velcro, or other secure attachment mechanism. Foot anchor 260 crosses over the talus and then wraps around the arch. Foot anchor 260 can be configured to connect to a shoe, boot, or directly to the foot.

The various straps and belts in FIGS. 10a-10c can be implemented using fabric or leather material, elastic bands, cables, or other suitable flexible connecting members. P2K 232 stores potential energy in the strapping arrangement, e.g., the elastic strapping stretches and stores potential energy when transitioning from a standing position to a crouched, squatting, or seated position. The potential energy in P2K 232 remains available while user 230 is seated to help with the opposing motion, i.e., standing up under load. When user 230 stands up from the seated position under load, similar to FIG. 2b, the potential energy in P2K 232 is released to assist the user in the motion. P2K 232 reduces the effort, strain, fatigue, and potential for injury associated with such movement in physical labor work environments.

FIGS. 11a-11c illustrate another embodiment of user 230 wearing P2K 232 with buckles 270 in side straps 252. Buckles 270 in combination with elastic material in side straps 252 provide further adjustments, support, and comfort for user 230 in donning, doffing, and wearing upper torso harness 240 and backpack assembly 242. Side straps 252 with buckles 250 keep back support 250 and seat webbing 254 in place during motion, e.g., while walking. Knee portion 258b includes connection point 272 to connect and separate a left half of the knee pad and a right half of the knee pad, which aids with donning and doffing leg strap arrangement 258. For example, user 230 brings the left half of the knee pad and the right half of the knee pad together and secures with connection point 272. To remove leg strap arrangement 258, user 230 detaches the left half of the knee pad from right half of the knee pad at connection point 272. Connection point 272 can be Velcro, belt, buckles, buttons, or other removable attachment mechanism.

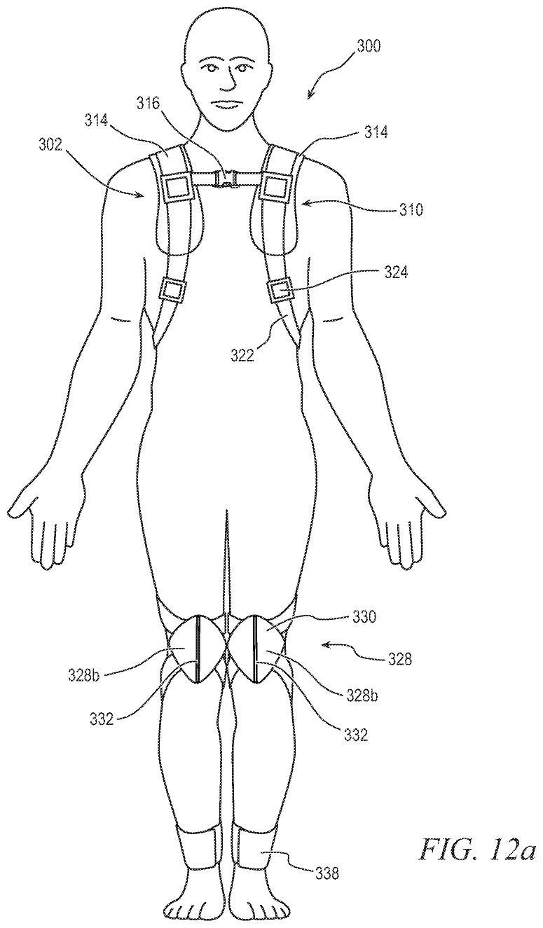

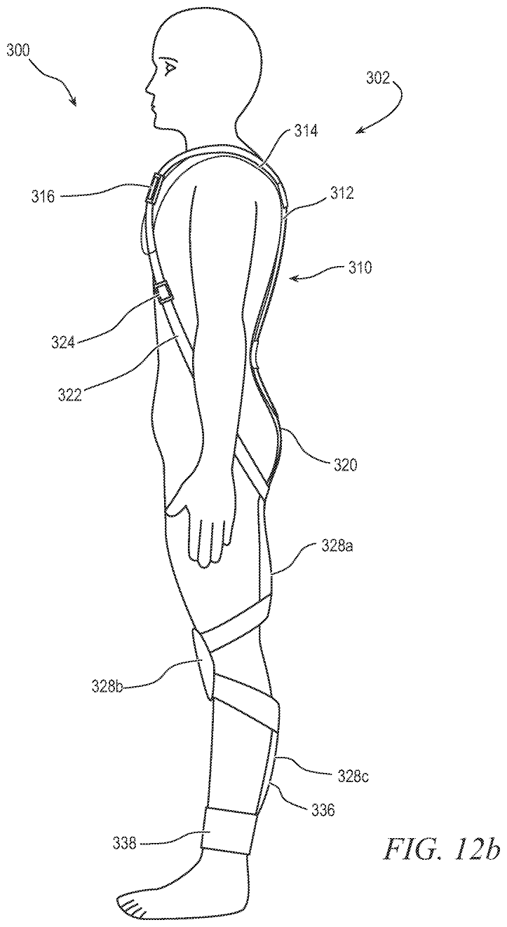

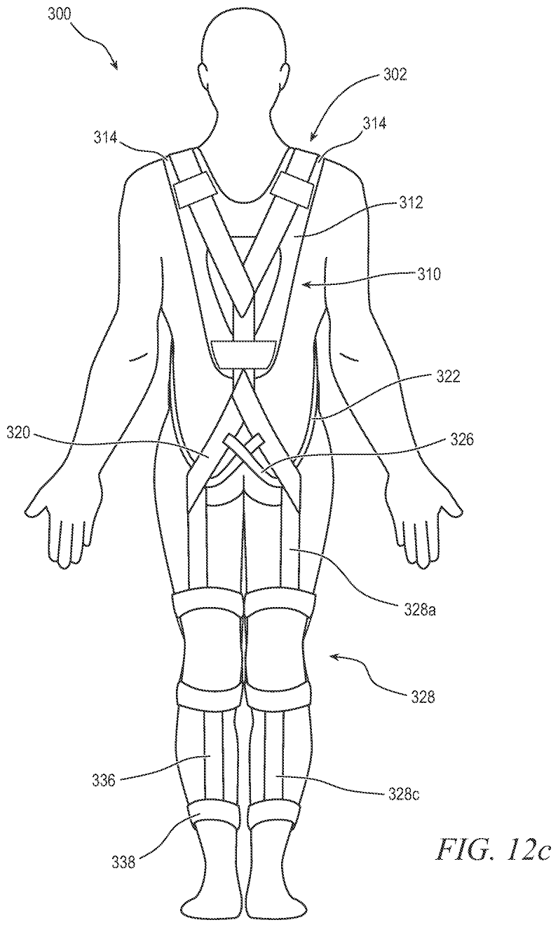

FIGS. 12a-12c illustrate another embodiment of user 300 wearing P2K 302 suitable for human assistance in crouching, squatting, and lifting under load. FIG. 12a shows a front view of P2K 302; FIG. 12b shows a side view of P2K 302; FIG. 12c shows a back view of P2K 302. P2K 302 is a passive personal augmentation suit or strapping system, i.e., no active components, applicable to many types of human activity and physical work, such as moving heavy articles, up and down motion under load, and other repetitive, long-term physical exertion. P2K 302 is capable of storing potential energy in the strapping arrangement, e.g., potential energy is stored in elastic strapping when transitioning from a standing position to a crouched or squatting position. The potential energy in P2K 302 is released when user 300 moves from the crouched or squatting position to return upright in stance, thereby assisting the user in the motion, particularly while under load. P2K 302 increases endurance, reduces fatigue, and decreases potential for injury associated with such activity, particularly for the user's back, legs, and knees. P2K 302 includes an interconnected arrangement of straps, belts, and braces to provide passively loaded support and reaction for user 300 while assisting with human motion under load. Portions of P2K 302 can be reflective material for safety.

Upper torso harness 310 includes backpack assembly 312 with shoulder straps 314. Backpack assembly 312 can include a combination of durable materials such as textured nylon, polyurethane coated polyester, and rigid plastic or polymer inserts, as well as elastic material and webbing material. Backpack assembly 312 can be made with an air mesh material with openings to allow for air-flow on the back and reduce the overall weight. Backpack assembly 312 covers a portion of back area of user 300 to improve ergonomics and comfort, relieve lower back pressure, and ease of donning and doffing. Shoulder straps 314 extend over the shoulder and affix to backpack assembly 312 with sewing, rivets, belt, buckles, buttons, snaps, hooks, adhesive, Velcro, or other secure attachment mechanism. Shoulder straps 314 are made with elastic material or webbing material and can slide, loosen, or tighten relative to backpack assembly 312 for user comfort and adapting to movement. Buckle 316 in shoulder straps 314 provides adjustment and attachment points. Buckle 316 snaps to lock and enable P2K 302 to fit different body sizes. Backside strap arrangement 320 extends over the gluteal area and connects to backpack assembly 312 with sewing, rivets, belt, buckles, buttons, snaps, hooks, adhesive, Velcro, or other secure attachment mechanism. Backside strap arrangement 320 slides or moves relative to backpack assembly 312 to enable or minimize medial/lateral relative motion and provide pre-tensioning. Shoulder straps 314 anchor to backpack assembly 312, and side straps 322 extend from backpack assembly 312 under the arms and along the side torso of user 300 to backpack assembly 312 or backside strap arrangement 320. Side straps 322 can include elastic material with buckles 324 for adjustments and comfort.

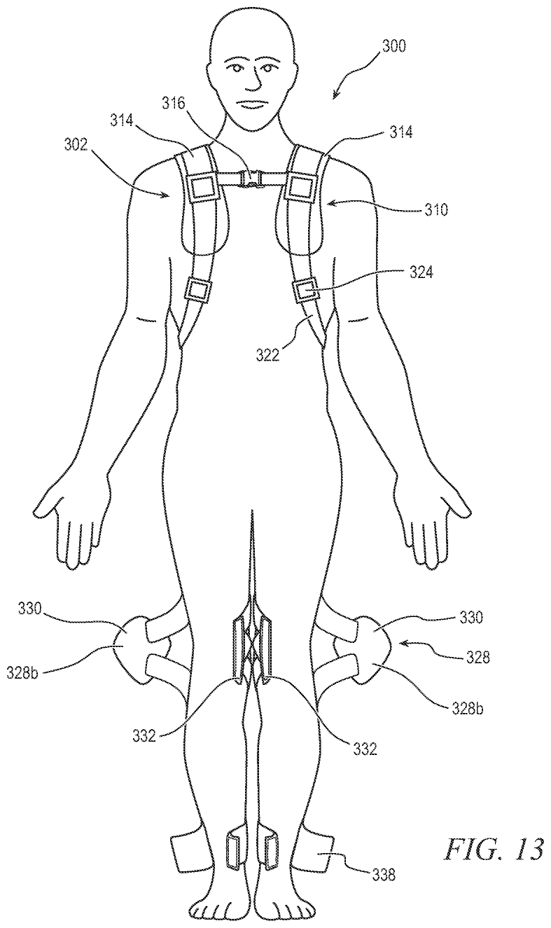

Leg strap arrangement 328 extends down the legs of user 300. Leg straps 328 include hamstring portion 328a over the hamstrings of user 300, knee portion 328b over the front of the knee area, and calf portion 328c over the calf area. Hamstring portion 328a connects to backside strap arrangement 320 with sewing, rivets, belt, buckles, buttons, snaps, hooks, adhesive, Velcro, or other secure attachment mechanism. Seat webbing 326 is connected within backside strap arrangement 320 with sewing, rivets, belt, buckles, buttons, snaps, hooks, adhesive, Velcro, or other secure attachment mechanism. Seat webbing 326 is an elastic material for storing and releasing power and comfortable load distribution. Knee portion 328b includes knee pads 330 for padding and protection of the knee while kneeling on the ground. Knee portion 328B provides an anchor point during energy storage and return while using P2K 302. In particular, knee pad 330 opens or splits along vertical or angled seam 332 and closes with buttons, snaps, hooks, Velcro, or other secure attachment mechanism for ease of donning and doffing P2K 302. Leg straps 328 utilize the bending motion of the knee for maximum stretching of the elastic bands. Calf portion 328c includes strap 336 extending down the tibia and strap cuff 338 over the calf. Strap 338 closes with buttons, hooks, snaps, Velcro, cuff, or other secure attachment mechanism. FIG. 13 shows knee pad 330 and strap cuff 338 in the open position. FIG. 12a shows knee pad 330 and strap cuff 338 in the closed position. The combination of shoulder strap buckle 316, split knee pads 330, and calf strap cuff 338 provide for ease of donning and doffing P2K 302.

The various straps and belts in FIGS. 12a-12c can be implemented using fabric or leather material, elastic bands, cables, or other suitable flexible connecting members. P2K 302 stores potential energy in the strapping arrangement, e.g., the elastic strapping stretches and stores potential energy when transitioning from a standing position to a crouched, squatting, or seated position. The potential energy in P2K 302 remains available while user 300 is seated to help with the opposing motion, i.e., standing up under load. When user 300 stands up from the seated position under load, similar to FIG. 2b, the potential energy in P2K 302 is released to assist the user in the motion. P2K 302 reduces the effort, strain, fatigue, and potential for injury associated with such movement in physical labor work environments.

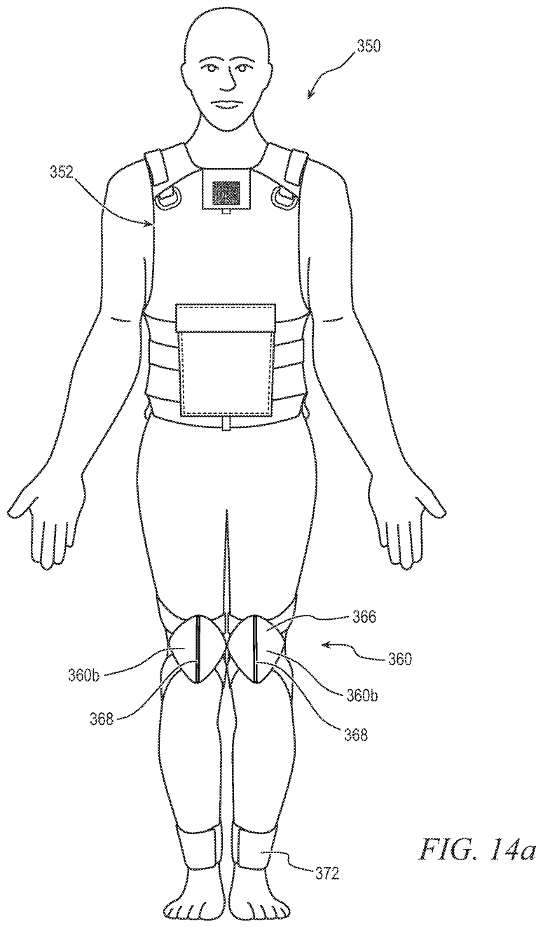

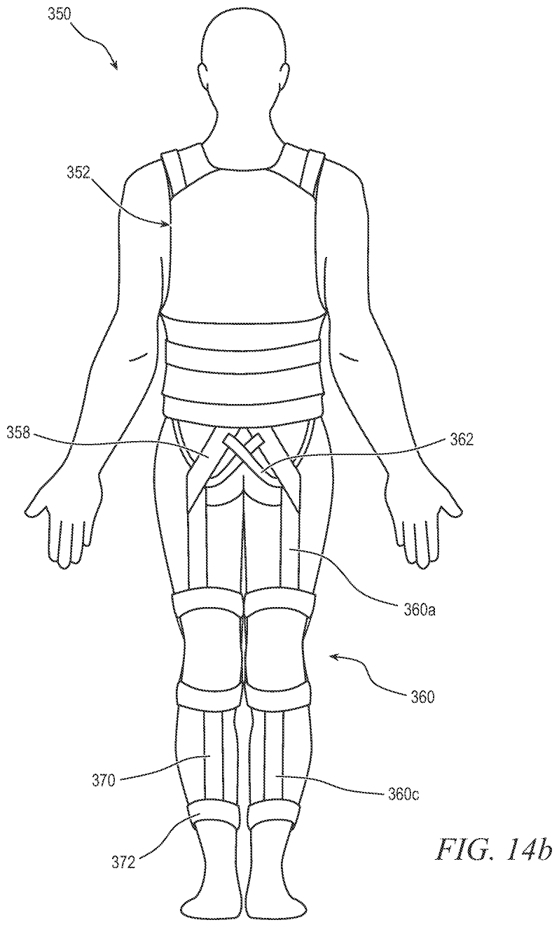

FIGS. 14a-14b illustrate an embodiment of P2K 350 suitable for human assistance in crouching, squatting, and lifting under load. P2K 350 is a passive personal augmentation suit or strapping system, i.e., no active components, applicable to many types of human activity and physical work, such as moving heavy articles, up and down motion under load, and other repetitive, long-term physical exertion. P2K 350 is capable of storing potential energy in the strapping arrangement, e.g., potential energy is stored in elastic strapping when transitioning from a standing position to a crouched or squatting position. The potential energy in P2K 350 is released when the user moves from the crouched or squatting position to return upright in stance, thereby assisting the user in the motion, particularly while under load. P2K 350 increases endurance, reduces fatigue, and decreases potential for injury associated with such activity, particularly for the user's back, legs, and knees. P2K 350 includes an interconnected arrangement of straps, belts, and braces to provide passively loaded support and reaction for the user while assisting with human motion under load. Portions of P2K 350 can be reflective material for safety.

P2K 350 includes a backpack assembly implemented as improved outer tactical vest (IOTV) or outer tactical vest (OTV) 352 connected to leg strap arrangement 360. IOTV/OTV 350 includes molle pouches capable of carrying personal armor protection, ammunition, and other tactical gear for military, law enforcement, and personal protection. Backside strap arrangement 358 extends over the gluteal area and connects to IOTV/OTV 350 with sewing, rivets, belt, buckles, buttons, snaps, hooks, adhesive, Velcro, or other secure attachment mechanism. Backside strap arrangement 358 slides or moves relative to IOTV/OTV 350 to enable or minimize medial/lateral relative motion and provide pre-tensioning. Leg strap arrangement 360 extends down the legs of the user. Leg straps 360 include hamstring portion 360a over the hamstrings of the user, knee portion 360b over the front of the knee area, and calf portion 360c over the calf area. Hamstring portion 360a connects to backside strap arrangement 358 with sewing, rivets, belt, buckles, buttons, snaps, hooks, adhesive, Velcro, or other secure attachment mechanism. Seat webbing 362 is connected within backside strap arrangement 358 with sewing, rivets, belt, buckles, buttons, snaps, hooks, adhesive, Velcro, or other secure attachment mechanism. Seat webbing 362 is an elastic material for storing and releasing power and comfortable load distribution. Knee portion 360b includes knee pads 366 for padding and protection of the knee while kneeling on the ground. Knee portion 360b provides an anchor point during energy storage and return while using P2K 350. In particular, knee pad 366 opens or splits along vertical or angled seam 368 and closes with buttons, snaps, hooks, Velcro, or other secure attachment mechanism for ease of donning and doffing P2K 350. Leg straps 360 utilize the bending motion of the knee for maximum stretching of the elastic bands. Calf portion 360c includes strap 370 extending down the tibia and strap cuff 372 over the calf. Strap 372 closes with buttons, hooks, snaps, Velcro, cuff, or other secure attachment mechanism.

While one or more embodiments of the present invention have been illustrated in detail, the skilled artisan will appreciate that modifications and adaptations to those embodiments may be made without departing from the scope of the present invention as set forth in the following claims.

What is claimed:

1. A human motion assistance device, comprising:
an upper torso harness configured to attach to a user, the upper torso harness including:
(a) an over-shoulder strap,
(b) an under-arm strap connected to the over-shoulder strap, and
(c) a lower-back strap configured to extend from the over-shoulder strap over a gluteal area of the user; and
a leg strap arrangement coupled to the lower-back strap, the leg strap arrangement including:
(d) a hamstring strap coupled to the lower-back strap and configured to extend over a rear hamstring of the user,
(e) a knee pad coupled to the hamstring strap by at least two attachment locations and configured to completely cover a knee of the user with a vertical seam configured to open and separate a first portion of the knee pad and a second portion of the knee pad for donning and doffing on the user,
(f) a calf strap coupled to the knee pad by at least two attachment locations and configured to extend over a rear calf of the user, and
(g) a calf cuff coupled to the calf strap and configured to wrap around a calf of the user.

2. The human motion assistance device of claim 1, further including an adjustable buckle connecting the over-shoulder strap to the under-arm strap.

3. The human motion assistance device of claim 1, wherein the lower-back strap includes seat webbing configured to extend over the gluteal area of the user.

4. The human motion assistance device of claim 3, wherein the seat webbing includes an elastic material.

5. The human motion assistance device of claim 1, wherein the leg strap arrangement includes an elastic material.

6. The human motion assistance device of claim 1, further including a backpack assembly configured to be disposed between the lower-back strap and a back of the user.

7. A human motion assistance device, comprising:
an upper torso harness configured to attach to a user, the upper torso harness including:
(a) an over-shoulder strap, and
(b) a lower-back strap configured to extend from the over-shoulder strap over a gluteal area of the user; and
a leg strap arrangement coupled to the lower-back strap, the leg strap arrangement including:
(c) a hamstring strap coupled to the lower-back strap and configured to extend over a rear hamstring of the user,
(d) a knee pad coupled to the hamstring strap and configured to cover a knee of the user with a vertical seam to open and separate a first portion of the knee pad and a second portion of the knee pad,
(e) a calf strap coupled to the knee pad and configured to extend over a rear calf of the user, and
(f) a calf cuff coupled to the calf strap and configured to wrap around a calf of the user.

8. The human motion assistance device of claim 7, further including:
an under-arm strap connected to the over-shoulder strap; and
an adjustable buckle connecting the over-shoulder strap to the under-arm strap.

9. The human motion assistance device of claim 7, wherein the lower-back strap includes seat webbing configured to extend over the gluteal area of the user.

10. The human motion assistance device of claim 9, wherein the seat webbing includes an elastic material.

11. The human motion assistance device of claim 7, wherein the leg strap arrangement includes an elastic material.

12. The human motion assistance device of claim 7, further including a backpack assembly configured to be disposed between the lower-back strap and a back of the user.

13. The human motion assistance device of claim 7, wherein the knee pad is coupled to the hamstring strap by at least two attachment locations.

14. A method of making a human motion assistance device, comprising:
providing an upper torso harness configured to attach to a user, the upper torso harness including:
(a) an over-shoulder strap, and
(b) a lower-back strap configured to extend from the over-shoulder strap over a gluteal area of the user; and
providing a leg strap arrangement coupled to the lower-back strap, the leg strap arrangement including:
(c) a hamstring strap coupled to the lower-back strap and configured to extend over a rear hamstring of the user,
(d) a knee pad coupled to the hamstring strap and configured to cover a knee of the user with a vertical seam to open and separate a first portion of the knee pad and a second portion of the knee pad,
(e) a calf strap coupled to the knee pad and configured to extend over a rear calf of the user, and
(f) a calf cuff coupled to the calf strap and configured to wrap around a calf of the user.

15. The method of claim 14, further including:
providing an under-arm strap connected to the over-shoulder strap; and
providing an adjustable buckle connecting the over-shoulder strap to the under-arm strap.

16. The method of claim 14, wherein the lower-back strap includes seat webbing configured to extend over the gluteal area of the user.

17. The method of claim 16, wherein the seat webbing includes an elastic material.

18. The method of claim 14, wherein the leg strap arrangement includes an elastic material.

19. The method of claim 14, further including disposing a backpack assembly between the lower-back strap and a back of the user.

20. The method of claim 14, wherein the knee pad is coupled to the hamstring strap by at least two attachment locations.
